SEQUENCE LISTING (2 sequences)

SEQ ID NO: 1 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQTAQARLGADMEDVCGRLVQYRGEVQAMLGQSTEELRVRLASHLRKLRKRLLRDPDDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 2 - protein, 300 aa, Homo sapiens
AKVEQAVETEPEPELRQQTEWQSGQRWELALGRFWDYLRWVQTLSEQVQEELLSSQVTQELRALMDETMKELKAYKSELEEQLTPVAEETRARLSKELQTAQARLGADMEDVCGRLVQYRGEVQAMLGQSTEELRVRLASHLRKLRKRLLRDPDDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

(19) United States
(12) Patent Application Publication
(10) Pub. No.: US 2009/0061457 A1
(43) Pub. Date: Mar. 5, 2009

(54) Apolipoprotein E3 protein as a biomarker of Parkinson's disease

(75) Inventors: Ira L. Goldknopf, The Woodlands, TX (US); Essam A. Sheta, The Woodlands, TX (US); Jennifer K. Bryson, The Woodlands, TX (US)

Correspondence Address: Power3 Medical Products, Inc., 3400 Research Forest Drive, The Woodlands, TX, 77381, US

(73) Assignee: Power3 Medical Products, Inc.

(21) Appl. No.: 11/899,212

(22) Filed: Sep. 5, 2007

(51) Int. Cl. G01N 33/68; C12Q 1/02; G01N 33/53

(52) U.S. Cl. 435/7.1; 435/29; 436/86; 436/501

(57) ABSTRACT

The present invention relates to an Apolipoprotein E3 protein as a biomarker for neurodegenerative disease, including Parkinson's disease, and the related diseases. More specifically, the present invention relates to the identification of an Apolipoprotein E3 protein, useful for the screening, diagnosis, and differentiation of Parkinson's disease from Alzheimer's disease, other neurodegenerative diseases, and normal controls.

### CROSS REFERENCE TO RELATED APPLICATIONS

This application claims priority to U.S. Utility patent application Ser. No. 11/503,881 filed Aug. 14, 2006 and entitled “Assay for Differentiating Alzheimer's and Alzheimer's Like Disorders” by inventors Ira L. Goldknopf et al. It also claims priority to U.S. Provisional Patent Application Ser. No. 60/708,992 filed Aug. 17, 2005 and entitled “Assay for Differentiating Alzheimer's and Alzheimer's Like Disorders” by inventors Ira L. Goldknopf et al. It also claims priority to U.S. Utility patent application Ser. No. 11/507,337 filed Aug. 21, 2006 and entitled “Assay for Diagnosis and Therapeutics Employing Similarities and Differences in Blood Serum Concentrations of 3 forms of Complement C3c and Related Protein Biomarkers between Amyotrophic Lateral Sclerosis and Parkinson's Disease” by inventors Ira L. Goldknopf et al.

### BACKGROUND OF THE INVENTION

1. Field of the Invention

The invention relates to the identification of a biomarker for the detection of neurodegenerative disease. More particularly, the present invention relates to the identification of an Apolipoprotein E3 protein as a biomarker useful in the screening, diagnosis, and differential diagnosis of Parkinson's disease (PD), Parkinson's disease Like (PD-Like) disorders, and Alzheimer's disease.

2. Description of the Related Art

Proteomics is a new field of medical research wherein proteins are identified and linked to biological functions, including roles in a variety of disease states. With the completion of the mapping of the human genome, the identification of unique gene products, or proteins, has increased exponentially. In addition, molecular diagnostic testing for the presence of certain proteins already known to be involved in certain biological functions has progressed from research applications alone to use in disease screening and diagnosis for clinicians. However, proteomic testing for diagnostic purposes remains in its infancy. There is, however, a great deal of interest in using proteomics for the elucidation of potential disease biomarkers.

Detection of abnormalities in the genome of an individual can reveal the risk or potential risk for individuals to develop a disease. The transition from such risk to the emergence of disease can be characterized as an expression of genomic abnormalities in the proteome. Thus, the appearance of abnormalities in the proteome signals the beginning of the process of cascading effects that can result in the deterioration of the health of the patient. Therefore, detection of proteomic abnormalities at an early stage is desirable in order to allow for detection of disease either before it is established or in its earliest stages where treatment may be most effective.

Recent progress using a novel form of mass spectrometry called surface enhanced laser desorption and ionization time of flight (SELDI-TOF) for the testing of ovarian cancer has led to an increased interest in proteomics as a diagnostic tool (Petrocoin, E. F. et al. 2002. Lancet 359:572-577). Furthermore, proteomics has been applied to the study of breast cancer through use of 2D gel electrophoresis and image analysis to study the development and progression of breast carcinoma in patients (Kuerer, H. M. et al. 2002. Cancer 95:2276-2282). In the case of breast cancer, breast ductal fluid specimens were used to identify distinct protein expression patterns in bilateral matched pair ductal fluid samples of women with unilateral invasive breast carcinoma.

Detection of biomarkers is an active field of research. For example, U.S. Pat. No. 5,958,785 discloses a biomarker for detecting long-term or chronic alcohol consumption. The biomarker disclosed is a single biomarker and is identified as an alcohol-specific ethanol glycoconjugate. U.S. Pat. No. 6,124,108 discloses a biomarker for mustard chemical injury. The biomarker is a specific protein band detected through gel electrophoresis and the patent describes use of the biomarker to produce protective antibodies or in a kit to identify the presence or absence of the biomarker in individuals who may have been exposed to mustard poisoning. U.S. Pat. No. 6,326,209 discloses measurement of total urinary 17 ketosteroid-sulfates as biomarkers of biological age. U.S. Pat. No. 6,693,177 discloses a process for preparation of a single biomarker specific for 0-2 acetylated sialic acid and useful for diagnosis and outcome monitoring in patients with lymphoblastic leukemia.

Neurodegenerative diseases such as Parkinson's disease (PD) are difficult to diagnose, particularly in their earlier stages. Currently there are no biomarkers available for either early diagnosis or use as drug targets for treatment of neurodegenerative diseases such as PD.

Therefore, there remains a need for better ways to detect and diagnose PD and to selectively distinguish it from other neurodegenerative diseases.

### SUMMARY OF THE INVENTION

The present invention relates to an Apolipoprotein E3 protein as a biomarker for neurodegenerative disease, whereby the concentration of an Apolipoprotein E3 protein in the serum of PD patients is significantly lower than age-matched control subjects. In addition, the concentrations of an Apolipoprotein E3 protein in the serum of patients with Alzheimer's disease (AD), and with PD-Like and Mixed disorders are significantly lower than age-matched controls and significantly higher than patients with Parkinson's disease.

One aspect of the present invention is the use of the biomarker, an Apolipoprotein E3 protein, for screening, diagnosis, or differential diagnosis of PD comprising: obtaining a blood serum sample from a test subject; determining the quantity of an Apolipoprotein E3 protein in the blood serum sample; and determining the ranges of the quantity of an Apolipoprotein E3 protein in blood serum samples from normal control individuals, from patients with PD, with AD, and with PD-Like and Mixed disorders, whereby the quantity of an Apolipoprotein E3 protein in the blood serum sample of the test subject within the range of PD values is indicative of the presence of PD, and the quantity of an Apolipoprotein E3 protein in the blood serum sample of the test subject outside the range of PD values is indicative of the absence of PD and the presence of a normal condition, or another neurological disorder, such as AD, or a PD-Like or Mixed disorder, such as: Frontotemporal dementia (FTD); Lewy body dementia (LBD); Alcohol related dementia; Semantic dementia; Vascular (Multi-infarct) dementia; Stroke (CVA); Post-irradiation Encephalopathy and Seizures; Vascular (Multi-Infarct) Parkinsonism; Idiopathic Sensory Ataxia; Corticalbasal Ganglionic Degeneration (CBGD); Multiple System Atrophy (MSA); Alzheimer's disease combined with Vascular (Multi-Infarct) dementia; Alzheimer's disease combined with Lewy body dementia; Parkinson's disease combined with Lewy body dementia; Alzheimer's and Parkinson's disease combined with Lewy body dementia; Frontotemporal dementia combined with Chronic Inflammatory Demyelinating Polyneuropathy; Thalamic CVA combined with HX of Lung CA; and Multiple System Atrophy combined with Subdural Hematoma.

Yet another aspect of the present invention is the use of the biomarker, an Apolipoprotein E3 protein, for differential diagnosis, or for screening of PD, comprising: obtaining a blood serum sample from a test subject; determining the quantity of an Apolipoprotein E3 protein in the blood serum sample; and determining the ranges the quantity of an Apolipoprotein E3 protein in blood serum samples from normal control individuals, from patients with PD, with AD, and with PD-Like and Mixed disorders, by two-dimensional gel electrophoresis; quantitating an Apolipoprotein E3 protein in the protein expression pattern; whereby the quantity of an Apolipoprotein E3 protein in the blood serum sample of the test subject within the range of PD values is indicative of the presence of PD, and the quantity of an Apolipoprotein E3 protein in the blood serum sample of the test subject outside the range of PD values is indicative of the absence of PD and the presence of a normal condition, or another neurological disorder, such as AD, or a PD-Like or Mixed disorder, such as: Frontotemporal dementia (FTD); Lewy body dementia (LBD); Alcohol related dementia; Semantic dementia; Vascular (Multi-infarct) dementia; Stroke (CVA); Post-irradiation Encephalopathy and Seizures; Vascular (Multi-Infarct) Parkinsonism; Idiopathic Sensory Ataxia; Corticalbasal Ganglionic Degeneration (CBGD); Multiple System Atrophy (MSA); Alzheimer's disease combined with Vascular (Multi-Infarct) dementia; Alzheimer's disease combined with Lewy body dementia; Parkinson's disease combined with Lewy body dementia; Alzheimer's and Parkinson's disease combined with Lewy body dementia; Frontotemporal dementia combined with Chronic Inflammatory Demyelinating Polyneuropathy; Thalamic CVA combined with HX of Lung CA; and Multiple System Atrophy combined with Subdural Hematoma.

Yet another aspect of the present invention is the use of the biomarker, an Apolipoprotein E3 protein, for differential diagnosis, or for screening of PD, comprising: obtaining a blood serum sample from a test subject; determining the quantity of an Apolipoprotein E3 protein in the blood serum sample; and determining the ranges the quantity of an Apolipoprotein E3 protein in blood serum samples from normal control individuals, from patients with PD, with AD, and with PD-Like and Mixed disorders; by an immunoassay using an antibody that recognizes an Apolipoprotein E3 protein; whereby the quantity of an Apolipoprotein E3 protein in the blood serum sample of the test subject within the range of PD values is indicative of the presence of PD, and the quantity of an Apolipoprotein E3 protein in the blood serum sample of the test subject outside the range of PD values is indicative of the absence of PD and the presence of a normal condition, or another neurological disorder, such as AD, or a PD-Like or Mixed disorder, such as: Frontotemporal dementia (FTD); Lewy body dementia (LBD); Alcohol related dementia; Semantic dementia; Vascular (Multi-infarct) dementia; Stroke (CVA); Post-irradiation Encephalopathy and Seizures; Vascular (Multi-Infarct) Parkinsonism; Idiopathic Sensory Ataxia; Corticalbasal Ganglionic Degeneration (CBGD); Multiple System Atrophy (MSA); Alzheimer's disease combined with Vascular (Multi-Infarct) dementia; Alzheimer's disease combined with Lewy body dementia; Parkinson's disease combined with Lewy body dementia; Alzheimer's and Parkinson's disease combined with Lewy body dementia; Frontotemporal dementia combined with Chronic Inflammatory Demyelinating Polyneuropathy; Thalamic CVA combined with HX of Lung CA; and Multiple System Atrophy combined with Subdural Hematoma.

The foregoing has outlined rather broadly several aspects of the present invention in order that the detailed description of the invention that follows may be better understood. Additional features and advantages of the invention will be described hereinafter which form the subject of the claims of the invention. It should be appreciated by those skilled in the art that the conception and the specific embodiment disclosed might be readily utilized as a basis for modifying or redesigning the structures for carrying out the same purposes as the invention. It should be realized by those skilled in the art that such equivalent constructions do not depart from the spirit and scope of the invention as set forth in the appended claims.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 illustrates the differentially expressed proteins detected in a 2D gel of blood serum collected from a patient, where the indicated protein spot (Protein spot N3314) is estimated to have MW of 34 KD and pI 6.2 by 2D gel electrophoresis, and is identified by LC-MS/MS of an in-gel trypsin digest of the spot as an Apolipoprotein E3 protein.

FIG. 2 is a comparative statistical Box and Whiskers graph (constructed using Analyze-it software for Microsoft Excel), illustrating the differential expression level of an Apolipoprotein E3 protein (spot N3314) in blood serum, based on the data from:

23 normal control individuals (Controls),

24 Parkinson's disease patients (PD),

44 Alzheimer's disease patients (AD), and

29 patients with PD-Like and Mixed disorders, including:
Frontotemporal dementia (FTD),
Lewy body dementia (LBD),
Alcohol related dementia,
Semantic dementia,
Vascular (Multi-infarct) dementia,
Stroke (CVA),
Post-irradiation Encephalopathy, Seizures,
Vascular (Multi-Infarct) Parkinsonism,
Idiopathic Sensory Ataxia,
Corticalbasal Ganglionic Degeneration (CBGD),
Multiple System Atrophy (MSA),
Alzheimer's disease combined with Vascular (Multi-Infarct) dementia,
Alzheimer's disease combined with Lewy body dementia,
Parkinson's disease combined with Lewy body dementia,
Alzheimer's and Parkinson's disease combined with Lewy body dementia,
Frontotemporal dementia combined with Chronic Inflammatory Demyelinating Polyneuropathy,
Thalamic CVA combined with HX or Lung CA, and
Multiple System Atrophy combined with Subdural Hematoma.

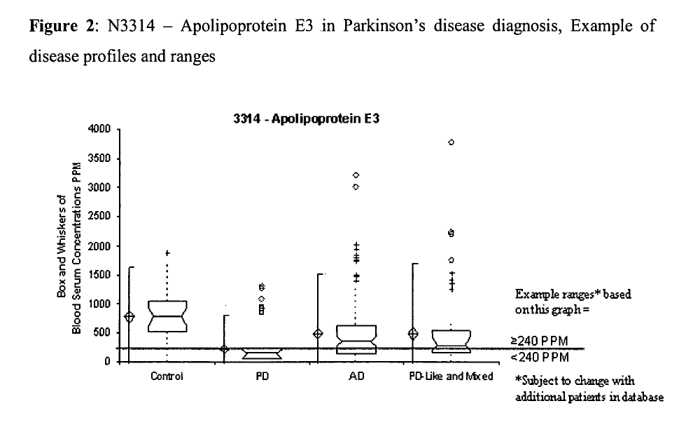

Also depicted in FIG. 2 are example concentration ranges, based on the data presented in the graph, for the purpose of illustrating preferred embodiments of the invention, including:

The concentration range of 0-239 PPM, where this range would correspond to the concentrations of Apolipoprotein E3 protein spot N3314 of individuals who have PD.

The concentration range of =240 PPM, where this range would correspond to the concentrations of Apolipoprotein E3 protein spot N3314 of patients who are normal or who have another neurological disorder, such as AD, or a PD-Like or Mixed disorder, such as: Frontotemporal dementia (FTD); Lewy body dementia (LBD); Alcohol related dementia; Semantic dementia; Vascular (Multi-infarct) dementia; Stroke (CVA); Post-irradiation Encephalopathy and Seizures; Vascular (Multi-Infarct) Parkinsonism; Idiopathic Sensory Ataxia; Corticalbasal Ganglionic Degeneration (CBGD); Multiple System Atrophy (MSA); Alzheimer's disease combined with Vascular (Multi-Infarct) dementia; Alzheimer's disease combined with Lewy body dementia; Parkinson's disease combined with Lewy body dementia; Alzheimer's and Parkinson's disease combined with Lewy body dementia; Frontotemporal dementia combined with Chronic Inflammatory Demyelinating Polyneuropathy; Thalamic CVA combined with HX of Lung CA; and Multiple System Atrophy combined with Subdural Hematoma.

Table 1 depicts the reproducibility of quantitation in 2D gels whereby 9 replicate analyses were performed with an individual sample of bovine serum albumin, where the sample was separated by 2D gel electrophoresis into a characteristic set of 5 spots which were then subjected to quantitation. The raw density counts (Gaussian Peak Values) shown are the individual values, averages, standard deviations, % Coefficients of Variation, and the quantity of the protein in nanograms (ng) for each spot.

Table 2 illustrates the identification of the amino acid sequence of protein spot N3314 as an Apolipoprotein E3 protein.

Table 3 illustrates the identification of the amino acid sequence of protein spot N3314 as the full size Apolipoprotein E3 after trimming the signal peptide off the amino terminal end of the molecule.

Table 4 depicts the summary statistics for the example of differential expression of blood serum concentrations of Apolipoprotein E3 protein (spot N3314), depicted in FIG. 2, of the groups of 23 normal controls, 24 PD patients (PD), 44 AD patients and 29 patients with PD-Like and Mixed disorders including Frontotemporal dementia (FTD); Lewy body dementia (LBD); Alcohol related dementia; Semantic dementia; Vascular (Multi-infarct) dementia; Stroke (CVA); Post-irradiation Encephalopathy and Seizures; Vascular (Multi-Infarct) Parkinsonism; Idiopathic Sensory Ataxia; Corticalbasal Ganglionic Degeneration (CBGD); Multiple System Atrophy (MSA); Alzheimer's disease combined with Vascular (Multi-Infarct) dementia; Alzheimer's disease combined with Lewy body dementia; Parkinson's disease combined with Lewy body dementia; Alzheimer's and Parkinson's disease combined with Lewy body dementia; Frontotemporal dementia combined with Chronic Inflammatory Demyelinating Polyneuropathy; Thalamic CVA combined with HX of Lung CA; and Multiple System Atrophy combined with Subdural Hematoma. Statistical significance was measured using analysis of variance (ANOVA-P=0.05, as constructed using Analyze-it software for Microsoft Excel).

### DESCRIPTION OF THE PREFERRED EMBODIMENTS

The present invention relates to an Apolipoprotein E3 protein as a biomarker for Parkinson's disease (PD). More particularly, the present invention relates to the identification of an Apolipoprotein E3 protein as a biomarker useful for the detection, diagnosis, and differentiation of patients with PD, from normal individuals and patients with other neurological disorders that are not PD, including AD and PD like (PD-Like) disorders and Mixed disorders including Frontotemporal dementia (FTD); Lewy body dementia (LBD); Alcohol related dementia; Semantic dementia; Vascular (Multi-infarct) dementia; Stroke (CVA); Post-irradiation Encephalopathy and Seizures; Vascular (Multi-Infarct) Parkinsonism; Idiopathic Sensory Ataxia; Multiple System Atrophy (MSA); Alzheimer's disease combined with Vascular (Multi-Infarct) dementia; Alzheimer's disease combined with Lewy body dementia; Parkinson's disease combined with Lewy body dementia; Alzheimer's and Parkinson's disease combined with Lewy body dementia; Frontotemporal dementia combined with Chronic Inflammatory Demyelinating Polyneuropathy; Thalamic CVA combined with HX of Lung CA; and Multiple System Atrophy combined with Subdural Hematoma.

The method for identification of an Apolipoprotein E3 protein as a biomarker for neurodegenerative disease is based on the comparison of 2D gel electrophoretic images of serum obtained from human subjects with and without diagnosed PD.

2D gel electrophoresis has been used in research laboratories for biomarker discovery since the 1970's (Margolis J. et al. 1969, Nature. 1969 221: 1056-1057; Orrick, L. R. et al. 1973; Proc Nat'l Acad. Sci. USA. 70: 1316-1320; Goldknopf, I. L. et al. 1975, J Biol Chem. 250: 7182-7187; Goldknopf, I. L. et al. 1977, Proc Nat'l Acad Sci USA. 74: 5492-5495; O'Farrell, P. H. 1975, J. Biol. Chem. 250: 4007-4021; Anderson, L. 1977, Proc Nat'l Aced Sci USA. 74: 864-868; Klose, J. 1975, Human Genetic. 26: 231-243). In the past, this method has been considered highly specialized, labor intensive and non-reproducible. Only recently with the advent of integrated supplies, robotics, and software, combined with bioinformatics, has progression of this proteomics technique in the direction of diagnostics become feasible. The promise and utility of 2D gel electrophoresis is based on its ability to detect changes in protein expression and to discriminate protein isoforms that arise due to variations in amino acid sequence and/or post-synthetic protein modifications such as phosphorylation, ubiquitination, conjugation with ubiquitin-like proteins, acetylation, glycosylation, and proteolytic processing. These are important variables in cell regulatory processes that are differentially expressed in blood serum biomarkers in neurodegenerative diseases, including AD and PD, and ALS (Goldknopf, I. L. et al. U.S. Utility patent application Ser. No. 11/507,337, Goldknopf et al. 2006 Biochem. Biophys. Res. Commun. 342: 1034-1039; Sheta E. A. et al. 2006, Expert Rev. Proteomics 3: 45-62).

There are few comparable alternatives to 2DGE for tracking changes in protein expression patterns related to disease. The introduction of high sensitivity fluorescent staining, digital image processing and computerized image analysis has greatly amplified and simplified the detection of unique species and the quantification of proteins. By using known protein standards as landmarks within each gel run, computerized analysis can detect unique differences in protein expression and modifications between two samples from the same individual or between several individuals.

Proteins of interest can be excised from the gels and the proteins can then be identified by in-gel digestion and matrix assisted laser desorption time of flight mass spectroscopy (MALDI-TOF MS) based peptide mass fingerprinting and database searching, or liquid chromatography with tandem mass spectrometry partial sequencing of individual peptides (LCMS/MS).

The identification of an Apolipoprotein E3 protein as a biomarker of neurodegenerative disease was based on a comparison of the 2D gel electrophoretic images of serum samples obtained from 23 normal controls, 24 Parkinson's disease patients (PD), 44 Alzheimer's disease (AD) patients, and 29 patients with PD-Like and Mixed disorders including Frontotemporal dementia (FTD); Lewy body dementia (LBD); Alcohol related dementia; Semantic dementia; Vascular (Multi-infarct) dementia; Stroke (CVA); Post-irradiation Encephalopathy and Seizures; Vascular (Multi-Infarct) Parkinsonism; Idiopathic Sensory Ataxia; Corticalbasal Ganglionic Degeneration (CBGD); Multiple System Atrophy (MSA); Alzheimer's disease combined with Vascular (Multi-Infarct) dementia; Alzheimer's disease combined with Lewy body dementia; Parkinson's disease combined with Lewy body dementia; Alzheimer's and Parkinson's disease combined with Lewy body dementia; Frontotemporal dementia combined with Chronic Inflammatory Demyelinating Polyneuropathy; Thalamic CVA combined with HX of Lung CA; and Multiple System Atrophy combined with Subdural Hematoma.

### Sample Collection and Preparation

Sample collection and storage have been performed in many different ways depending on the type of sample and the conditions of the collection process. In the present study, serum samples were collected, aliquoted and stored in a −80° C. freezer before analysis format.

In a preferred embodiment of the invention, the serum samples were removed from 80° C. and placed on ice for thawing. To each 100 μL of sample, 100 μL of LB-2 buffer (7M urea. 2M Thiourea, 1% DTT, 1% Triton X-100, 1X Protease inhibitors, and 0.5% Ampholyte pH 3-10) was added and the mixture vortexed. The sample was incubated at room temperature for about 5 minutes.

### Two Dimensional Gel Electrophoresis of Samples

Separation of the proteins in the serum samples was then performed using 2D gel electrophoresis. The 2D gel electrophoretic images were obtained, compared and analyzed as described in the U.S. Provisional Patent Application Ser. No. 60/614,315 entitled “Differential Protein Expression Patterns Related to Disease States” filed Sep. 29, 2004 and incorporated herein by reference. A protein assay was performed on the sample to determine total protein content in μg.

Approximately 100 μg of the solubilized protein pellet was suspended in a total volume of 184 μL of IEF loading buffer containing 1 μL Bromophenol Blue as a marker to trace the progress of the electrophoresis. Each sample was loaded onto an 11 cm IEF strip (Bio-Rad), pH 5-8, and overlaid with 1.5-3.0 ml of mineral oil to minimize the sample buffer evaporation. Using the PROTEAN® IEF Cell, an active rehydration was performed at 50V and 20° C. for 12-18 hours.

IEF strips were then transferred to a new tray and focused for 20 min. at 250V followed by a linear voltage increase to 8000V over 2.5 hours. A final rapid focusing was performed at 8000V until 20,000 volt-hours were achieved. Running the IEF strip at 500V until the strips were removed finished the isoelectric focusing process.

Isoelectric focused strips were incubated on an orbital shaker for 15 mm with equilibration buffer (2.5 ml buffer/strip). The equilibration buffer contained 6M urea, 2% SDS, 0.375M HC1, and 20% glycerol, as well as freshly added DTT to a final concentration of 30 mg/ml. An additional 15 mm incubation of the IEF strips in the equilibration buffer was performed as before, except freshly added iodoacetamide (C2H4INO) was added to a final concentration of 40 mg/mil. The IPG strips were then removed from the tray using clean forceps and washed five times in a graduated cylinder containing the Bio Rad running buffer 1×Tris-Glycine-SDS.

The washed IEF strips were then laid on the surface of Bio Rad pre-cast CRITERION SDS-gels 8-16%. The IEF strips were fixed in place on the gels by applying a low melting agarose. A second dimensional separation was applied at 200V for about one hour. After electrophoresis, the gels were carefully removed and placed in a clean tray and washed twice for 20 minutes in 100 ml of pre-staining solution containing 10% methanol and 7% acetic acid.

### Staining and Analysis of the 2D Gels

The gels were stained with SYPRO RUBY (Bio-Rad Laboratories) and subjected to fluorescent digital image analysis. The protein patterns of the serum samples were analyzed using PDQUEST™ (Bio-Rad Laboratories) image analysis software.

The 2D gel patterns of the 23 serum samples collected from normal control subjects were compared with each other pursuant to the methodology described in the U.S. Utility patent application Ser. No. 11/172,219 entitled “Differential Protein Expression Patterns Related to Disease States” filed Sep. 29, 2004 and incorporated herein by reference. The 23 normal individual blood serum samples all gave similar 2D gel protein patterns.

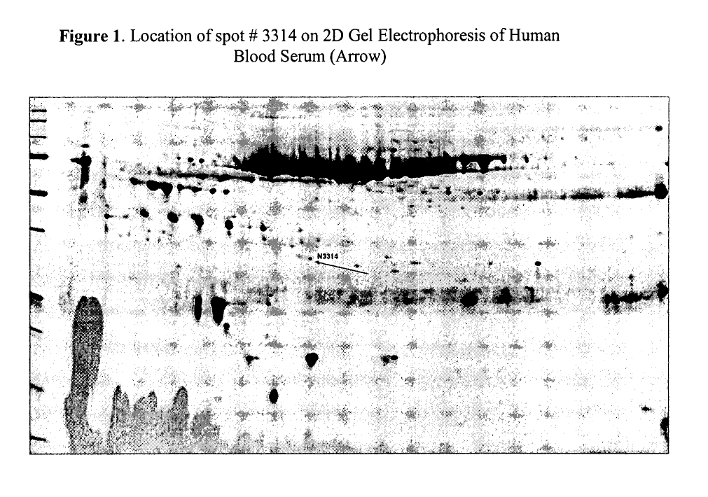

These normal protein expression patterns were then compared to the gel patterns obtained with blood serum samples from the 24 Parkinson's disease patients (PD), 44 Alzheimer's disease (AD) patients and 29 patients with PD-Like and Mixed disorders including or a PD-Like or Mixed disorder, such as: Frontotemporal dementia (FTD); Lewy body dementia (LBD); Alcohol related dementia; Semantic dementia; Vascular (Multi-infarct) dementia; Stroke (CVA); Post-irradiation Encephalopathy and Seizures; Vascular (Multi-Infarct) Parkinsonism; Idiopathic Sensory Ataxia; Corticalbasal Ganglionic Degeneration (CBGD); Multiple System Atrophy (MSA); Alzheimer's disease combined with Vascular (Multi-Infarct) dementia; Alzheimer's disease combined with Lewy body dementia; Parkinson's disease combined with Lewy body dementia; Alzheimer's and Parkinson's disease combined with Lewy body dementia; Frontotemporal dementia combined with Chronic Inflammatory Demyelinating Polyneuropathy; Thalamic CVA combined with HX of Lung CA; and Multiple System Atrophy combined with Subdural Hematoma. When the gel patterns of PD patients were compared to the gel patterns of normal subjects, protein spot 3314, of particular interest, was identified as shown in FIG. 1. Protein spot 3314 was selected for further investigation. Protein spot 3314 was quantitated by stain intensity in each of the normal and disease patient groups of serum samples.

In order to assess the reproducibility of the 2D gels and staining, 75 nanograms of bovine serum albumin (BSA) was run on 9 separate 2D gels. The gels were stained with SYPRO RUBY and the 5 spots resolved in the BSA region of the gel were then subjected to quantitative analysis using PDQUEST™ and the Gaussian Peak Value method. The results shown in Table 1 illustrate that the electrophoretic patterns were reproducible and the reproducibility (% Coefficient of Variation=% CV) was independent of the spot amount over the range tested (2.9-38.6 ng/spot).

TABLE 1

| | Spot # | | | | |
|---|---|---|---|---|---|
| Replicate # | 9901 | 9902 | 9904 | 9905 | 9906 |
| 1 | 332 | 1152 | 2612 | 739 | 229 |
| 2 | 246 | 974 | 2694 | 513 | 167 |
| 3 | 336 | 1065 | 2354 | 668 | 225 |
| 4 | 311 | 1272 | 3482 | 713 | 198 |
| 5 | 351 | 1168 | 2724 | 733 | 245 |
| 6 | 268 | 1059 | 2753 | 622 | 184 |
| 7 | 452 | 1630 | 4000 | 946 | 281 |
| 8 | 405 | 1195 | 2752 | 870 | 274 |
| 9 | 258 | 1050 | 2716 | 699 | 189 |
| AVG | 329 | 1174 | 2899 | 723 | 221 |
| STDEV | 68 | 193 | 510 | 127 | 40 |
| % CV | 21% | 16% | 18% | 18% | 18% |
| ng/spot | 4.4 | 15.6 | 38.6 | 9.6 | 2.9 |

Reproducibility of Quantitation in 9 Gels
PDQuest Gaussian Peak Value of the Major Components of BSA

### The Isolation and Identification of the Protein Spot N3314

Protein spot N4411 was carefully excised, in-gel digested with trypsin, and subjected to mass fingerprinting/sequence analysis by high performance liquid chromatography/tandem mass spectrometry (LC-MS/MS) and expert database searching.

Tandem mass spectrometry provides a powerful means of determining the structure and identity of proteins and peptides. The unknown tryptic peptide is first separated and purified by liquid chromatography and then the effluent from the separation is vaporized by electrospray, separated in a mass spectrometer and then bombarded with high-energy electrons causing it to fragment in a characteristic manner, indicative of its amino acid sequence. The fragments, which are of varying mass and charge, are then passed through a magnetic field and separated according to their mass/charge ratios. The resulting characteristic fragmentation pattern of the unknown peptide is used to identify its amino acid sequence.

A protein can often be unambiguously identified by an LC MS/MS analysis of its constituent peptides (produced by either chemical or enzymatic treatment of the sample).

Following differential expression analysis, protein spot N3314 was carefully excised from the gel for identification. Excised gel spots of protein N3314 were de-stained by washing the gel spots twice in 100 mM NH₄HCO₃ buffer, followed by soaking the gel spots in 100% acetonitrile for 10 minutes. The acetonitrile was aspirated before adding the trypsin solution.

Typically, a small volume of trypsin solution (approximately 5-15 μg/ml trypsin) is added to the de-stained gel spots and incubated at 3 hours at 37° C. or overnight at 30° C. The digested peptides were extracted, washed, desalted and subjected to liquid chromatography followed by tandem mass spectral analysis to identify protein spot N3314. Those of skill in the art are familiar with mass spectral analysis of digested peptides. The mass spectral analysis was conducted on a Micromass LC QTOF (Waters). Peptide fragmentation patterns were obtained from the tryptic in-gel digests of protein spot N3314 and the patterns were subjected to public database searches using the GenBank and dbEST databases maintained by the National Center for Biotechnology Information (hereinafter referred to as the NCBI database). Those of skill in the art are familiar with searching databases, such as the NCBI database. The NCBI database search results were displayed with the best matched amino acid sequences of the identified peptides and the protein accession of number the protein sequence they were derived from. For protein spot N3314, the protein identified by the NCBI database search was an Apolipoprotein E3 protein (Table 2).

Given the results of 2D gel electrophoresis, wherein the protein spot N3314 has a MW of 34 KD, and a pI of 6.2, it is most likely that the protein spot 3314 corresponds to the full size mature Apolipoprotein E3 after trimming the signal peptide off the amino terminal end of the molecule (Tables 2-3).

TABLE 2
Apolipoprotein E, [Includes N3314 = E3]

Alternative Names:
AD2; BROAD-BETALIPOPROTEINEMIA; FLOATING-BETALIPOPROTEINEMIA; MGC1571; apoprotein
APOE APOLIPOPROTEIN E, DEFICIENCY OR DEFECT OF
Alzheimer disease 2 (APOE*E4-associated, late onset)
CORONARY ARTERY DISEASE, SEVERE, SUSCEPTIBILITY TO
DYSBETALIPOPROTEINEMIA DUE TO DEFECT IN APOLIPOPROTEIN E-d
FAMILIAL HYPERBETA- AND PREBETALIPOPROTEINEMIA
FAMILIAL HYPERCHOLESTEROLEMIA WITH HYPERLIPEMIA
HYPERLIPEMIA WITH FAMILIAL HYPERCHOLESTEROLEMIC XANTHOMATOSIS
HYPERLIPOPROTEINEMIA, TYPE III
Apolipoprotein E
Apolipoprotein E precursor
Apolipoprotein E3
Amino Acid Sequence of Apolipoprotein E3 [N3314]: NCBI accession #178849:
LC/MS/MS identified peptides span underlined:

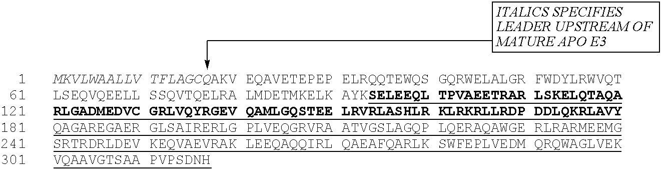

TABLE 3

The 2D gel estimated pI = 6.2 and MW = 34KD. The best fit to these data for amino acid sequence
of N3314 is:

AKVEQAVETEPEPELRQQTEWQSGQRWELALGRFWDYLRWVQTLSEQVQEELLSSQVTQELRALMDETMKELKAYKSELEEQLTPVAEETRARLS
KELQTAQARLGADMEDVCGRLVQYRGEVQAMLGQSTEELRVRLASHLRKIRKRLLRDPDDLQKRLAVYQAGAREGAERGLSAIRERLGPLVE
QGRVRAATVGSLAGQPLQERAQAWGERLRARMEEMGSRTRDRLDEVKEQVAEVRAKLEEQAQQIRLQAEAFQARLKSWFEPLVEDMQRQWAGLV
EKVQAAVGTSAAPVPSDNH
Protein: 5.5
MW: 34364

### Role of Apolipoprotein E3 in Neurodegenerative Disorders:

Apolipoprotein E3 binds to the NMDA receptors on the neurons, modulating Calcium influx. Low levels of Apolipoprotein E3 likely result in de-regulation of Calcium influx, subjecting the neurons in the substantia nigra of the brain of Parkinson's disease patients to oxidative stress related cell death (Sheta et al. 2006, Expert Review of Proteomics 3: 45-62) also due to a substantia nigra directed immune inflammatory response (He et al. 2002, Experimental Neurology 176: 322-327; Sheta et al. 2006). Apolipoprotein E3 has also been found localized in alpha-Synuclein containing neurofibrillary tangles associated with Parkinson's disease and Abeta containing plaques in Alzheimer's disease (Gee et al. 2005, J. Biochem. Cell Biol. 37: 1145-1150), which may account for the reduction of its concentration in blood serum in inclusion body related neurodegenerative diseases (Sheta et al. 2006).

### Serum Level of Apolipoprotein E3:

The blood serum concentrations of Apolipoprotein E3 protein spot N3314 were determined in 23 normal controls, 24 Parkinson's disease patients (PD), 44 Alzheimer's disease (AD) patients and 29 patients with PD-Like or Mixed disorders, including Frontotemporal dementia (FTD); Lewy body dementia (LBD); Alcohol related dementia; Semantic dementia; Vascular (Multi-infarct) dementia; Stroke (CVA); Post-irradiation Encephalopathy and Seizures; Vascular (Multi-Infarct) Parkinsonism; Idiopathic Sensory Ataxia; Corticalbasal Ganglionic Degeneration (CBGD); Multiple System Atrophy (MSA); Alzheimer's disease combined with Vascular (Multi-Infarct) dementia; Alzheimer's disease combined with Lewy body dementia; Parkinson's disease combined with Lewy body dementia; Alzheimer's and Parkinson's disease combined with Lewy body dementia; Frontotemporal dementia combined with Chronic Inflammatory Demyelinating Polyneuropathy; Thalamic CVA combined with HX of Lung CA; and Multiple System Atrophy combined with Subdural Hematoma. The blood serum of patients with PD is characterized by significantly low concentrations of Apolipoprotein E3 protein spot N3314, when compared to normal subjects, AD patients, and patients with PD-Like and Mixed disorders. In addition, the patients with AD or PD-Like or Mixed disorders are also characterized by significantly low blood serum concentrations of Apolipoprotein E3 protein spot N3314, when compared to normal controls, which are still significantly high when compared to PD patients (FIG. 2, Table 4).

As depicted in Table 4, the mean level of blood serum concentrations of Apolipoprotein E3 protein spot N3314 in the group of 23 normal control individuals was 774.4±52.10 S.E. (PPM).

Also depicted in Table 4, the mean level of blood serum concentrations of Apolipoprotein E3 protein spot N3314 in the group of 24 PD patients was 220.3±38.74 S.E. (PPM).

Also depicted in Table 4, the mean level of blood serum concentrations of Apolipoprotein E3 protein spot N3314 in the group of 44 patients with AD patients was 483.6±40.52 S.E. ppm.

Also depicted in Table 4, the mean level of blood serum concentrations of Apolipoprotein E3 protein spot N3314 in the group of 29 patients with PD-Like or Mixed disorder, such as: Frontotemporal dementia (FTD); Lewy body dementia (LBD); Alcohol related dementia; Semantic dementia; Vascular (Multi-infarct) dementia; Stroke (CVA); Post-irradiation Encephalopathy and Seizures; Vascular (Multi-Infarct) Parkinsonism; Idiopathic Sensory Ataxia; Corticalbasal Ganglionic Degeneration (CBGD); Multiple System Atrophy (MSA); Alzheimer's disease combined with Vascular (Multi-Infarct) dementia; Alzheimer's disease combined with Lewy body dementia; Parkinson's disease combined with Lewy body dementia; Alzheimer's and Parkinson's disease combined with Lewy body dementia; Frontotemporal dementia combined with Chronic Inflammatory Demyelinating Polyneuropathy; Thalamic CVA combined with HX of Lung CA; and Multiple System Atrophy combined with Subdural Hematoma, was 477.2±58.83 S.E. ppm.

### Model Application of the Apolipoprotein E3 as a Biomarker for the Differential Diagnosis of Parkinson's Disease

As depicted in Table 4, the blood serum concentration values of Apolipoprotein E3 protein spot N3314 for the population of the PD patients are significantly lower than those of the normal control individuals (P<0.0001), those of patients with AD (P<0.0001) and those of patients with PD-Like and Mixed disorders (P<0.0001), including patients diagnosed with a PD-Like or Mixed disorder, such as: Frontotemporal dementia (FTD); Lewy body dementia (LBD); Alcohol related dementia; Semantic dementia; Vascular (Multi-infarct) dementia; Stroke (CVA); Post-irradiation Encephalopathy and Seizures; Vascular (Multi-Infarct) Parkinsonism; Idiopathic Sensory Ataxia; Corticalbasal Ganglionic Degeneration (CBGD); Multiple System Atrophy (MSA); Alzheimer's disease combined with Vascular (Multi-Infarct) dementia; Alzheimer's disease combined with Lewy body dementia; Parkinson's disease combined with Lewy body dementia; Alzheimer's and Parkinson's disease combined with Lewy body dementia; Frontotemporal dementia combined with Chronic Inflammatory Demyelinating Polyneuropathy; Thalamic CVA combined with HX of Lung CA; and Multiple System Atrophy combined with Subdural Hematoma. Also, the blood serum concentration values of Apolipoprotein E3 protein spot N3314 of patients with AD, PD-Like and Mixed disorders were significantly lower than those of the normal controls (P<0.0001).

Thus, the differences in the blood serum concentrations of Apolipoprotein E3 protein spot N3314 between the patient groups all display high degrees of statistical significance (P<0.0001).

Hence, in one embodiment of the invention, the blood serum concentration of Apolipoprotein E3 protein is used in the differential diagnosis of Parkinson's disease.

For the purpose of illustrating this preferred embodiment of the invention, the example concentration ranges of Apolipoprotein E3 protein spot N3314 depicted in FIG. 2, based on the data presented in the graph, are used.

In another embodiment of the invention, the blood serum concentration of Apolipoprotein E3 protein spot N3314 is used to screen patients with movement disorder. symptoms for Parkinson's disease. In this embodiment, only two answers are sought: PD, indicating a high likelihood of PD being present; or not PD, indicating a high likelihood of PD not being present.

For the purpose of illustrating this preferred embodiment of the invention, the example concentration ranges of Apolipoprotein E3 protein spot N3314 depicted in FIG. 2, based on the data presented in the graph, are used.

TABLE 4
Summary statistics of Apolipoprotein E3-N3314 in
blood sera of Neurodegenerative diseases, indicated by
(a) Mean level (PPM) ± SE and (b) ANOVA statistics
(a)

| Groups | n | Mean | ± | SE |
|---|---|---|---|---|
| Control | 23 | 774.4 | ± | 52.10 |
| PD | 24 | 220.3 | ± | 38.74 |
| AD | 44 | 483.6 | ± | 40.52 |
| PD-Like and Mixed | 29 | 477.2 | ± | 58.83 |

(b)

| Statistically Significant Differences | ANOVA-P |
|---|---|
| Control vs. AD | <0.0001 |
| Control vs. PD | <0.0001 |
| Control vs. PD-Like and Mixed | <0.0001 |
| PD vs. AD | <0.0001 |
| PD vs. PD-Like and Mixed | <0.0001 |

The blood serum samples may also be subjected to various other techniques known in the art for separating and quantitating proteins. Such techniques include, but are not limited to: gel filtration chromatography, ion exchange chromatography, reverse phase chromatography, affinity chromatography (typically in an HPLC or FPLC apparatus), affinity capture, one dimensional gel or capillary electrophoresis, or any of the various centrifugation techniques well known in the art. Certain embodiments would also include a combination of one or more chromatography; electrophoresis or centrifugation steps combined via electrospray or nanospray with mass spectrometry or tandem mass spectrometry of the proteins themselves, or of a total digest of the protein mixtures. Certain embodiments may also include surface enhanced laser desorption mass spectrometry or tandem mass spectrometry, or any protein separation technique that determines the pattern of proteins in the mixture, either as a one-dimensional, two-dimensional, three-dimensional or multi-dimensional protein pattern, and/or the pattern of protein post synthetic modifications or different isoforms of an Apolipoprotein E3 protein are used.

Quantitation of a protein by antibodies directed against that protein is well known in the field. The techniques and methodologies for the production of one or more antibodies to an Apolipoprotein E3 protein are routine in the field and are not described in detail herein.

As used herein, the term antibody is intended to refer broadly to any immunologic binding agent such as IgG, 1 gM, IgA, IgD and IgE. Generally, IgG and/or 1 gM are preferred because they are the most common antibodies in the physiological situation and because they are most easily made in a laboratory setting.

Monoclonal antibodies (MAbs) are recognized to have certain advantages, e.g., reproducibility and large-scale production, and their use is generally preferred. The invention thus provides monoclonal antibodies of human, murine, monkey, rat, hamster, rabbit, chicken, or other animal origin. Due to the ease of preparation and ready availability of reagents, murine monoclonal antibodies are generally preferred. However, human auto antibodies or “humanized” antibodies are also contemplated, as are chimeric antibodies from mouse, rat, or other species, bearing human constant and/or variable region domains, bispecific antibodies, recombinant and engineered antibodies and fragments thereof.

The term “antibody” thus also refers to any antibody-like molecule that has a 20 amino acid antigen binding region, and includes antibody fragments such as Fab′, Fab, F(ab′)2, single domain antibodies (DABS), Fv, scFv (single chain Fv), and the like. The techniques for preparing and using various antibody-based constructs and fragments are well known in the art. Means of preparing and characterizing antibodies are also well known in the art (See, e.g., Antibodies: A Laboratory Manual, Cold Spring Harbor Laboratory, 1988; incorporated herein by reference).

Antibodies to an Apolipoprotein E3 protein may be used in a variety of assays in order to quantitate the protein in serum samples, or other fluid or tissue samples. Well known methods include immunoprecipitation, antibody sandwich assays, ELISA and affinity chromatography methods that include antibodies bound to a solid support. Such methods also include micro arrays of antibodies or proteins contained on a glass slide or a silicon chip, for example.

It is contemplated that arrays of antibodies to an Apolipoprotein E3 protein, or peptides derived from an Apolipoprotein E3 protein, may be produced in an array and contacted with the serum samples or protein fractions of serum samples in order to quantitate the blood serum concentrations of an Apolipoprotein E3 protein. The use of such micro arrays is well known in the art and is described, for example in U.S. Pat. No. 5,143,854, incorporated herein by reference.

The present invention includes a screening assay for neurodegenerative disease based on the up-regulation and/or down-regulation of an Apolipoprotein E3 protein expression. One embodiment of the assay will be constructed with antibodies to an Apolipoprotein E3 protein. One or more antibodies targeted to antigenic determinants of an Apolipoprotein E3 protein will be spotted onto a surface, such as a polyvinyl membrane or glass slide. As the antibodies used will each recognize an antigenic determinant of an Apolipoprotein E3 protein, incubation of the spots with patient samples will permit attachment of an Apolipoprotein E3 protein to the antibody.

The binding of an Apolipoprotein E3 protein can be reported using any of the known reporter techniques including radioimmunoassays (RIA), stains, enzyme linked immunosorbant assays (ELISA), and sandwich ELISAs with a horseradish peroxidase (HRP)-conjugated second antibody also recognizing an Apolipoprotein E3 protein, the pre-binding of fluorescent dyes to the proteins in the sample, or biotinylating the proteins in the sample and using an HRP-bound streptavidin reporter. The HRP can be developed with a chemiluminescent, fluorescent, or calorimetric reporter. Other enzymes, such as luciferase or glucose oxidase, or any enzyme that can be used to develop light or color can be utilized at this step.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods, and in the steps or in the sequence of steps of the methods described herein without departing from the concept, spirit and scope of the invention.

More specifically, it is well recognized in the art that the statistical data, including but not limited to the mean, standard error, standard deviation, median, interquartile range, 95% confidence limits, results of analysis of variance, non-parametric median tests, discriminant analysis, etc., will vary as data from additional patients are added to the database or antibodies are utilized to determine concentrations of an Apolipoprotein E3 protein or any biomarker. Therefore changes in the range of concentrations of an Apolipoprotein E3 protein do not depart from the concept, spirit and scope of the invention.

Also more specifically, it is disclosed (in cross referenced U.S. Utility patent applications by Goldknopf, I. L. et al. Ser. Nos. 11/507,337 and 11/503,881, U.S. Provisional Patent Applications by Goldknopf et al. Ser. No. 60/708,992 and Ser. No. 60/738,710, and referenced in Goldknopf, I. L et al. 2006 and E. A. Sheta et al, 2006, hereby incorporated as reference) that blood serum concentrations of protein biomarkers, including Apolipoprotein E3 protein spot N3314, can be used in combination with other biomarkers for diagnosis, differential diagnosis, and screening. Consequently, the use of an Apolipoprotein E3 protein in conjunction with one or more additional biomarkers does not depart from the concept, spirit and scope of the invention.

It is also well recognized in the art that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

It is also well recognized in the art that there are other Non-Parkinson's neurological disorders related to those already mentioned that are hereby included within the scope of the invention including but not limited to Atypical parkinsonism, Ataxia, Dystonia, Progressive Supranuclear Palsy, Essential tremor, Mild Cognitive Impairment, Amyotrophic Lateral Sclerosis, and any neurological disease or disorder, injury, depression or other psychiatric condition, or any other PD-Like disorder with symptoms similar to Parkinson's disease that results from any other cause.

### Application Project Computer Readable Amino Acid Sequence Listing

### <120> Title: AN APOLIPOPROTEIN E3 PROTEIN AS A BIOMARKER OF PARKINSON'S DISEASE

### <130> App File Reference: 3314 PD

### <140> Current App Number:

### <141> Current Filing Date: ______

### Earlier Applications

<150> Prior App Number: U.S. Ser. No. 11/503,881

### <151> Prior Filing Date: 2006-08-14

### Earlier Applications

### <150> Prior App Number: U.S. Provisional 60/708,992

### <151> Prior Filing Date: 2005-08-17

### Earlier Applications

<150> Prior App Number: U.S. Ser. No. 11/507,337

### <151> Prior Filing Date: 2006-08-21

### Organization Applicant

Street: 3400 Research Forest Drive

City: The Woodlands

State: Texas

Country: USA

Postal Code: 77381

Phone Number:

Fax Number:

Email Address:

### <110> Organization Name: Power3 Medical Products, Inc.

### Individual Applicant

Street: 42 Brushwood Court

City: The Woodlands

State: Texas

Country: USA

Postal Code: 77380

Phone Number: 281-466-1600

Fax Number: 281-466-1481

Email Address: igoldknopf@power3medical.com

### <110> Last Name: Goldknopf

### <110> First Name: Ira

### <110> Middle Initial: L.

### <110> Suffix: Ph.D.

### Individual Applicant

Street: 71 Merryweather Circle

City: The Woodlands

State: Texas

Country: USA

Postal Code: 77384

Phone Number: 281-466-1600

Fax Number: 281-466-1481

Email Address: esheta@power3medical.com

### <110> Last Name: Sheta

### <110> First Name: Essam

### <110> Middle Initial: A

### <110> Suffix: Ph.D.

### Individual Applicant

Street: 26001 Budde Road, #2801

City: Spring

State: Texas

Country: USA

Postal Code: 77380

Phone Number: 281-466-1600

Fax Number: 281-466-1481

Email Address: jbryson@power3medical.com

### <110> Last Name: Bryson

### <110> First Name: Jennifer

### <110> Middle Initial: K

### <110> Suffix:

### Individual Applicant

Street:

City: Houston

State: Texas

Country: USA

Postal Code:

Phone Number: 281-466-1600

Fax Number:

Email Address:

### <110> Last Name: Stanley

### <110> First Name: Appel

### <110> Middle Initial: H

### <110> Suffix: M.D.

### Sequence 1

<213> Organism Name: Homo sapiens

### <400> Pre Sequence String:

| | |
|---|---|
| MKVLWAALLV TFLAGCQAKV EQAVETEPEP ELRQQTEWQS GQRWELALGR | 60 |
| FWDYLRWVQT | |
| LSEQVQEELL SSQVTQELRA LMDETMKELK AYKSELEEQL TPVAEETRAR | 120 |
| LSKELQTAQA | |
| RLGADMEDVC GRLVQYRGEV QAMLGQSTEE LRVRLASHLR KLRKRLLRDP | 180 |
| DDLQKRLAVY | |
| QAGAREGAER GLSAIRERLG PLVEQGRVRA ATVGSLAGQP LQERAQAWGE | 240 |
| RLRARMEEMG | |
| SRTRDRLDEV KEQVAEVRAK LEEQAQQIRL QAEAFQARLK SWFEPLVEDM | 300 |
| QRQWAGLVEK | |
| VQAAVGTSAA PVPSDNH | 317 |
| <212> Type: PRT | |
| <211> Length: 317 | |

Sequence Name Apolipoprotein E3 Pre protein

Sequence Description Amino acid sequence, accession #178849, of the precursor to Apolipoprotein E3 protein spot N3314 containing the leader sequence, amino acids 1-17, the span of LC MS/MS identified tryptic peptides from in-gel digestion, amino acids 94-180, and amino acid sequences upstream and downstream of the peptide span, amino acids 18-93 and 181-317, respectively.

### Sequence 2

<213> Organism Name: Homo sapiens

### <400> Pre Sequence String:

| | |
|---|---|
| AKVEQAVETE PEPELRQQTE WQSGQRWELA LGREWDYLRW VQTLSEQVQE | 60 |
| ELLSSQVTQE | |
| LRALMDETMK ELKAYKSELE EQLTPVAEET RARLSKELQT AQARLGADME | 120 |
| DVCGRLVQYR | |
| GEVQAMLGQS TEELRVRLAS HLRKLRKRLL RDPDDLQKRL AVYQAGAREG | 180 |
| AERGLSAIRE | |
| RLGPLVEQGR VRAATVGSLA GQPLQERAQA WGERLRARME EMGSRTRDRL | 240 |
| DEVKEQVAEV | |
| RAKLEEQAQQ IRLQAEAFQA RLKSWFEPLV EDMQRQWAGL VEKVQAAVGT | 300 |
| SAAPVPSDNH | |
| <212> Type: PRT | |
| <211> Length: 300 | |

Sequence Name Apolipoprotein E3

Sequence Description The mature Apolipoprotein E3 amino acid sequence, wherein the leader sequence is removed, leaving the rest of the protein intact (301 amino acids). This corresponds to the Apolipoprotein E3 protein spot N3314, with an amino acid sequence estimated pI of 5.5, and MW 34,364 KD, versus a 2D gel estimated pI of 6.2 and MW of 34 KD.

What is claimed is:

1. A biomarker for diagnosis, differential diagnosis and screening for a neurodegenerative disease comprising an Apolipoprotein E3 protein in a blood serum sample.

2. The biomarker of claim 1, wherein the neurodegenerative disease is Parkinson's disease.

3. The biomarker of claim 1, wherein the neurodegenerative disease is Alzheimer's disease.

4. The biomarker of claim 1, wherein the neurodegenerative disease is a Parkinson's disease like (PD-Like) or Mixed disorder, such as: Frontotemporal dementia (FTD); Lewy body dementia (LBD); Alcohol related dementia; Semantic dementia; Vascular (Multi-infarct) dementia; Stroke (CVA); Post-irradiation Encephalopathy and Seizures; Vascular (Multi-Infarct) Parkinsonism; Idiopathic Sensory Ataxia; Corticalbasal Ganglionic Degeneration (CBGD); Multiple System Atrophy (MSA); Alzheimer's disease combined with Vascular (Multi-Infarct) dementia; Alzheimer's disease combined with Lewy body dementia; Parkinson's disease combined with Lewy body dementia; Alzheimer's and Parkinson's disease combined with Lewy body dementia; Frontotemporal dementia combined with Chronic Inflammatory Demyelinating Polyneuropathy; Thalamic CVA combined with HX of Lung CA; and Multiple System Atrophy combined with Subdural Hematoma.

5. The biomarker of claim 1, wherein the Apolipoprotein E3 protein includes one or more of the amino acid sequences in Tables 2 and 3.

6. The biomarker of claim 1, wherein the Apolipoprotein E3 protein includes one or more antigenic determinants of the Apolipoprotein E3 protein, located within one or more of the amino acid sequences in Tables 2 and 3.

7. The use of the biomarker of claim 1 in a method for screening, diagnosing and/or differentially diagnosing for a neurodegenerative disease comprising:
obtaining a blood, blood serum, or blood plasma sample from a test subject; determining a quantity of an Apolipoprotein E3 protein in the subject sample; and comparing the quantity of an Apolipoprotein E3 protein in the test subject sample with ranges of values of the quantity of an Apolipoprotein E3 protein in samples of normal control subjects; and one or more groups of patients with a neurodegenerative disease,
whereby a quantity of an Apolipoprotein E3 protein in the test subject sample is indicative of a neurodegenerative disease or a normal condition.

8. The method of claim 7, wherein the quantity of an Apolipoprotein E3 protein is determined by two-dimensional gel electrophoresis.

9. The method of claim 8, wherein the two-dimensional gel electrophoresis comprises a separation by isoelectric point followed by a separation by molecular weight.

10. The method of claim 8, wherein the two-dimensional gel is stained and an intensity of the biomarker of claim 1 is proportional to the expression of the biomarker of claim 1 in the serum sample.

11. The method of claim 7, wherein the quantity of an Apolipoprotein E3 protein is determined by one or more antibodies to one or more antigenic determinants of an Apolipoprotein E3 protein, located within one or more of the amino acid sequences in Tables 2 and 3.

12. The method of claim 7 wherein the quantity of an Apolipoprotein E3 protein is determined by one or more of a number of protein quantitative fractionation techniques, including but not limited to gel filtration chromatography, ion exchange chromatography, reverse phase chromatography, affinity chromatography, affinity capture, or 1 dimensional gel or capillary electrophoresis.

13. The method of claim 7 wherein the ranges of blood serum concentrations of an Apolipoprotein E3 protein in any group of normal controls or neurodegenerative diseases is determined by statistics.

14. The method of claim 7 wherein the quantity of an Apolipoprotein E3 proteins determined along with the quantity of one or more other biomarkers for diagnosis, differential diagnosis or screening for a neurodegenerative disease.

15. The method of claim 7, wherein the screening, diagnosis or differential diagnosis is an adjunct to at least one other diagnostic test for the neurodegenerative disease.

16. The method of claim 11 wherein the quantity of an Apolipoprotein E3 protein in the subject sample is determined by transferring the protein from a one or two-dimensional gel to a PVDF membrane (Western blot) and contacting the transferred protein with at least one antibody with reactivity to the amino acid sequences in Table 2 and 3.

17. The method of claim 11 wherein the quantity of an Apolipoprotein E3 protein in the subject sample is determined by any type of immunoassay techniques.
